(19) United States
(12) Patent Application Publication
(10) Pub. No.: US 2026/0024613 A1
(43) Pub. Date: Jan. 22, 2026

(54) MULTI-OMICS TENSOR REGRESSION FOR COMPLEX DISEASES

(71) Applicant: International Business Machines Corporation, Armonk, NY (US)

(72) Inventors: ARITRA BOSE, Astoria, NY (US); Myson Burch, West Harrison, NY (US); Laxmi Parida, Mohegan Lake, NY (US)

(21) Appl. No.: 18/776,947

(22) Filed: Jul. 18, 2024

(51) Int. Cl. G16B 20/00; G16B 40/00

(52) U.S. Cl. CPC G16B 20/00; G16B 40/00

(57) ABSTRACT

Provided are methods, systems and computer program product embodiments for analyzing multi-omic data using a tensor regression model for genome-wide association studies in the life sciences. The unique structure of tensor covariates is leveraged to find associations between the omics data and complex diseases. Within this framework, the excessive dimensionality is reduced to a manageable level, leading to efficient estimations and predictions. The method is superior to using classical regression techniques in genome-wide association studies, which are challenged by analyzing multi-dimensional and uniquely structured data from the health and life sciences, in which covariates can take on more intricate forms such as multi-dimensional arrays. Embodiments have multiple uses in genomics, proteomics, metabolomics, multi-omics data integration, drug discovery, personalized medicine and predictive modeling, demonstrating the versatility and importance of tensor regression models to understand the associations between omics data and complex diseases.

### BACKGROUND

### 1. Technical Field

Present invention embodiments relate to analyzing multiple biological data types (“omes”) from several health- or life-sciences sources using tensor regression. More specifically, embodiments of the invention relate to finding associations between the multi-omics data, at least some of which have high dimensionality and intricate structures, and complex diseases, leading to efficient estimations and predictions that have the potential to lead to advancements in several areas in healthcare and biomedical research.

Classical regression techniques handle a covariate as a vectors and calculate a corresponding vector of regression coefficients. In contemporary applications in health and life sciences, covariates take on more intricate forms, such as high-order, multi-dimensional arrays (i.e., tensors) with complex structures. Conventional statistical and computational approaches fall short in analyzing these high-throughput data due to their extremely high dimensionality and intricate structures, which cannot be captured by the conventional vector-based or matrix-based regression models. One such approach is a genome-wide association study (GWAS), which can be restrictive in many ways, finding only single genetic marker associations with the phenotypes of interest. The results from Genome-wide association studies alone are more limited when complex, multi-omics data are used, but data from genome-wide association studies can be used together with the results of tensor regression analysis to provide insights into diagnoses, treatment and prognoses for complex diseases.

### 2. Discussion of the Related Art

Tensor regression has recently been used in the analysis of genetic data and neural networks but none of this work focuses on tensor regression of multi-omics data described in this disclosure. In particular, some of this work focuses on uncovering gene networks from tensor representations of gene expression experiments, or relates to the use of individual-SNP data for revealing population structure. Other work applied sketching based on randomized algorithms on tensor regressions, or addressed shortcomings of deep neural networks when manipulating high-order tensors using tensor contraction and tensor decomposition, essentially improving on neural network approaches. Still other work involved tensor regression in the context of computational complexity for joint modeling of gene-gene interactions, but no previous work has modeled multi-omic data through tensor regression modeling, and combined that with genome-wide association studies data or used it in genome-wide association studies to inform on development of, or treatments or prognoses for, complex diseases?

### SUMMARY

According to one embodiment of the present invention, multi-omics data are analyzed through a tensor regression model in genome-wide associations studies to identify associations between these data and complex diseases; the analysis takes place by using a processor at each of at least the following steps: (i) performing data pre-processing and quality control across each modality; (ii) combining modalities into higher-order tensors; and (iii) computing associations using a tensor regression model. The multi-omics data are analyzed through a tensor regression model to yield associations for use in genome-wide association studies to inform clinicians and researchers about complex diseases.

### BRIEF DESCRIPTION OF THE DRAWINGS

Generally, like reference numerals in the various figures are utilized to designate like components.

FIG. 1 is a diagrammatic illustration of an example of a computing environment according to an embodiment of the present invention.

FIG. 2, according to an embodiment of the present invention, depicts a high-level view of the process of analyzing multi-omics data in a tensor regression model for use in genome-wide association studies to inform clinicians and researchers about complex diseases.

FIG. 3 depicts another embodiment of the process of FIG. 2.

FIG. 4 is a flow chart identifying the primary steps of the genome-wide association study with linear or logistics regression modeling, for example, as a comparison to the process analyzing multi-omics data using tensor regression modeling and interpreting those results in genome-wide association studies.

FIG. 5 is a flow chart identifying steps in the tensor regression process.

FIG. 6 describes the proof of concept with 70% accuracy for the use of dual omics data in a tensor regression model in genome-wide association studies.

FIG. 7 lists several advantages an embodiment of the present invention exhibits over the use of classical regression models in genome-wide association studies in the health and life sciences.

FIG. 8 lists several broad uses of an embodiment of the present invention in the health and life sciences areas.

### DETAILED DESCRIPTION

The treatment of complex diseases requires a comprehensive understanding of the patient and their history from multi-modal data set spanning across electronic medical records (EMRs) to their molecular profiling from whole genomic, transcriptome, proteome sequencing to imaging data from many timepoints, often referred to as multi-omics.

This disclosure provides an innovative approach to association studies involving tensor regression models that effectively leverage the unique structure of tensor covariates to find associations between omics data and complex diseases. Within this framework, excessive dimensionality of the data is reduced to a manageable level, known as decomposition, leading to efficient estimations and predictions. Thus, provided herein is an approach to fast and scalable genome-wide association studies using tensor regression.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

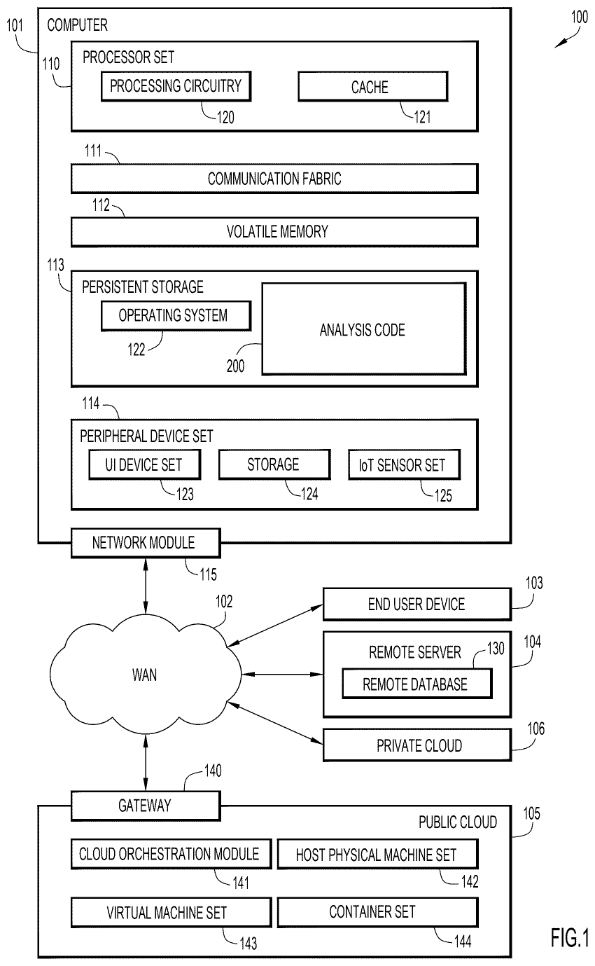

Referring to FIG. 1, computing environment 100 contains an example of an environment for the execution of at least some of the computer code involved in performing the inventive methods, such as analysis code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in

FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

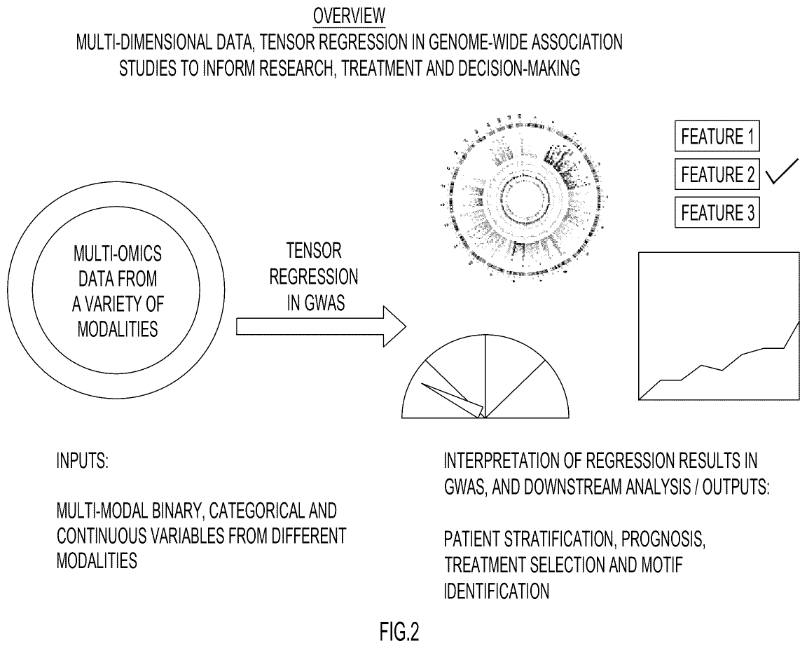

A overview of genome-wide association studies using multi-omics data in tensor regression (e.g., via analysis code 200, computer 101, etc.) according to an embodiment of the present invention is illustrated in FIG. 2.

FIG. 2 provides an overview of using tensor regression in genome-wide association studies, including identifying various categories of data inputs such as multi-model binary, categorical, and continuous variables from different modalities and multiple sources of data, such as proteomics, metabolomics, clinical and medical records, imaging data, EEG and EKG records, other human and veterinary clinical records, and other health- or life-sciences related records, among others. These data can include multi-dimensional, and complex data such as, for example, magnetic resonance imaging (MRI) 3- or 4-dimensional brain images, multichannel EEG data, and metabolic syndrome-related clinical data such as cholesterol, hemoglobin A1c, triglycerides, body mass index, c-reactive protein, etc. Robust tensor regression data are interpreted in genome-wide association studies. Outputs are wide-ranging and include identification of associations between modalities and complex disease outcomes, for example, evaluating potential biomarkers for accelerating therapeutic development. This allows patient stratification, prognosis, treatment selection and motif identification, and further enables, for example, treatment of subjects using one or more therapeutic agents developed as a result of identification of key associations between complex data and diseases. Outputs and uses of the method of an embodiment of the present invention are discussed in more depth below.

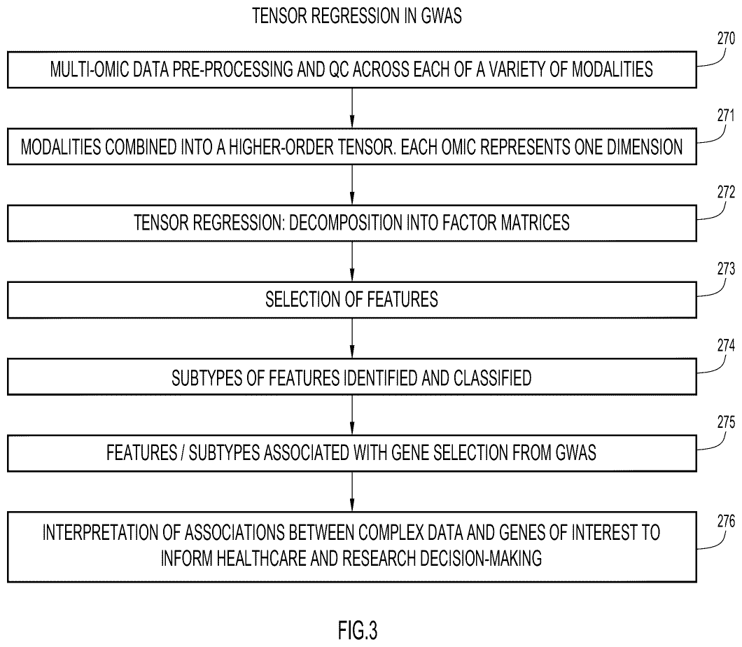

Turning to FIG. 3, which depicts a method (e.g., implemented via analysis code 200, computer 101, etc.) of the genome-wide association studies involving use of multi-omics data in a tensor regression model 270-271 including decomposition 272 and further downstream analysis including selection of features 273, and identification and classification of subtypes of features 274, followed by associating gene selections from the association studies with the subtypes and classifications of features 275. Finally, the outputs of the regression and downstream analyses 275 are interpreted based on the associations between the complex data and the genes of interest to inform both healthcare and research decision-making 276. Further downstream analysis also will inevitably yield new therapeutic, including pharmaceutical-based, approaches to the treatment of subjects having complex diseases. Various types of decomposition may be used as a part of the tensor regression processing, including but not limited to Tucker decomposition, CP decomposition and spike tensor decomposition.

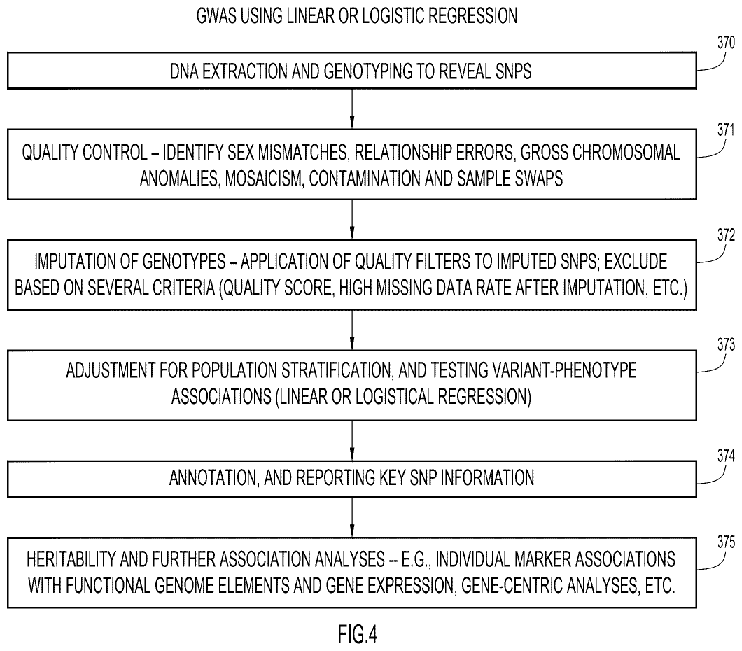

Turning to FIG. 4, the primary steps of the previously known genome-wide association studies in which genotyping data (single nucleotide polymorphisms, SNPs) are a type of unidimensional data inputted and associated with genes of interest in the studies. Data are initially collected by extracting DNA from an individual's blood sample for genotyping analysis in which SNPs are identified and mapped 370. Quality control measures 371 are carried out to identify sex mismatches and various other anomalies. Following quality control, filters are applied 372 to imputed SNP data and certain data may be excluded at this step based on a number of criteria such as quality score, high number of missing data rates, etc. Adjustments 373 are made for population stratification and variant-phenotype associations are tested through linear or logistic regression models; finally, annotation, SNP detail reporting 374, heritability and further association analyses 375 are undertaken to inform about gene expression and other end points. This figure is provided as a comparison to the embodiment shown in FIG. 3, which utilizes multiple-dimensional data run through a tensor regression model in genome-wide association studies.

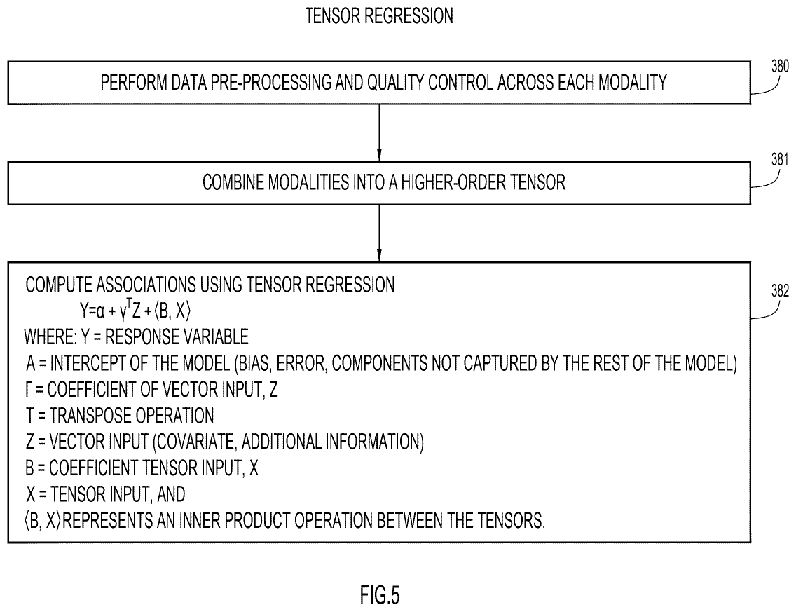

The general steps of tensor regression (e.g., performed via analysis code 200, computer 101, etc.) are illustrated in FIG. 5. The method comprises data pre-processing and quality control across each modality 380; combining modalities into a tensor 381; and computing associations using tensor regression 382. In another embodiment of the use of tensor regression in genome-wide association studies, the high dimensionality of multi-omics data is reduced through tensor regression by using sparse random projections to approximately obtain the coefficients across multi-omics modalities.

Step 380 of FIG. 5 relates to pre-processing and quality control of data, meaning the data are reviewed for anomalies, contamination, etc.

Step 381 of FIG. 5 involves combining modalities of the inputted data, some or all may be multi-dimensional or have intricate shapes, into a higher-ordered tensor. For example, data encompassed in an image may include various dimensions relating to pixels, spacing of pixels, color and transparency of pixels, or other aspects of the image, etc.

Step 382 of FIG. 5 relates to computing associations using the tensor regression model (I):

$$Y = α + {γ}^{T} Z + 〈 B , X 〉$$

where Y represents the response (i.e., dependent) variable;
α represents the intercept of the model (i.e., bias, error, or a component not captured by the rest of the model);
γ represents a coefficient vector corresponding to Z;
T represents a transpose operation for γ;
Z represents the vector input (covariates, additional information);
B represents the coefficient tensor corresponding to X; and
X represents the tensor input.
   Variations on the tensor regression model (I) are well known and may be used in various embodiments of the invention, depending on the type and density of the data, and the number of samples and omics. A skilled artisan would understand that essentially any other version of the tensor regression model may be used in an embodiment of the present invention, as tensor regression in general previously was known in the art. In certain other embodiments, dimensionality reduction is performed by using sparse random projections to approximate coefficients across multi-omics modalities.

The embodiment of FIG. 3 may utilize the tensor regression model of FIG. 5, step 382, or any another version of the tensor regression model discussed above.

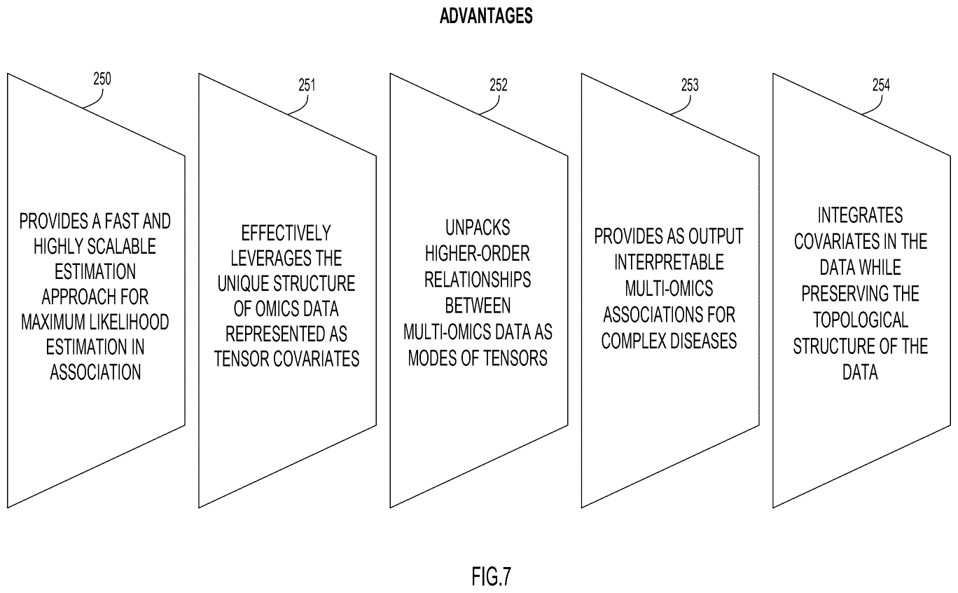

FIG. 7 depicts several advantages of an embodiment of the present invention exhibits over classical regression techniques for analyzing multi-omics data in the life and health sciences. These include the provision of a highly scalable estimation approach 250 for maximum likelihood estimation in association; the ability to leverage 251 the unique structure of omics data, represented as a tensor covariate; the identification of higher order relationships between multi-omics data as modes of tensors 252; the provision of interpretable multi-omics associations for complex diseases 253; and the integration of the covariates in the data while preserving the topological structure of the data 254. The term “integration” in general refers to the combination and evaluation of all data that are processed. In healthcare, integration usually refers to the process of merging and consolidating healthcare information from diverse sources.

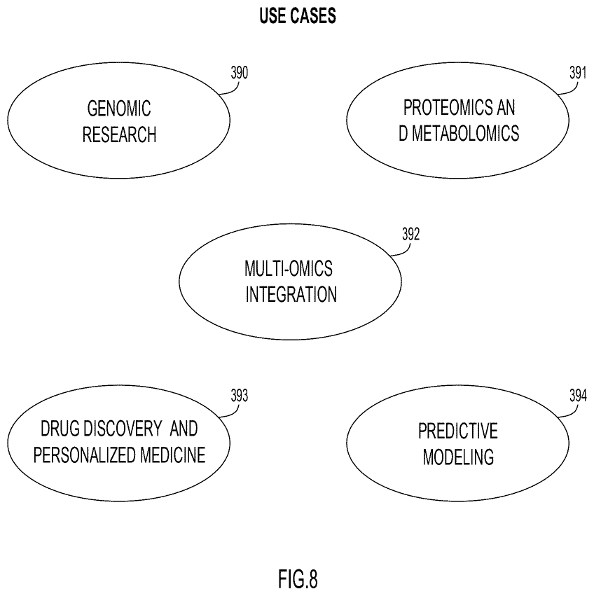

FIG. 8 depicts a number of possible use cases for any number of embodiments of the present invention. These include genomics research 390, proteomics and metabolomics 391, multi-omics integration 392; drug discovery and personalized medicine 393, and predictive modeling 394. With regard to genomics research 390, the present invention embodiment is capable of identifying genetic variants associated with complex diseases; for example, it can be used for evaluating gene-gene interactions (epistasis) in relation to disease susceptibility. It also can detect gene-environmental interactions and their impact on disease risk. With respect to proteomics and metabolics 391, the present invention embodiment can uncover associations between protein or metabolite profiles and disease outcomes; it can also be used for investigating and developing biomarkers for use in research, or in clinical practice in disease diagnosis, prognosis or treatments. With regard to multi-omics integration 392, the present invention embodiment integrates data from multiple omics sources (for example, genomics, proteomics, metabolomics, medical records, imaging results, etc., in the human clinical, research or veterinary medicine fields, among other life science areas) to gain a comprehensive understanding of complex diseases; it can also be used to identify cross-omics associations and interactions. With respect to drug discovery and personalized medicine 393, the present invention embodiment can be used to identify potential drug targets by linking omics data with disease-related factors, and to develop personalized treatment strategies based on individual omics profiles. Finally, with respect to predictive modeling 394, the present invention embodiment can be used for developing predictive models for disease risk, progression, or treatment response based on omics data; it also can estimate patient outcomes and prognosis. These applications demonstrate the versatility and importance of tensor regression models in exploring and understanding the associations between omics data and complex diseases, ultimately having the potential to contribute to advancements in several areas in healthcare and biomedical research. An embodiment of the present invention may be used in human medical or in veterinary fields, including healthcare, research, drug discovery, personalized medicine and predictive modeling. Multi-omics data may derive from human clinical or veterinary sources.

Other embodiments of this disclosure include a computer program product for analyzing multi-omics data using tensor regression. The computer program product comprises one or more computer readable storage media having program instructions collectively stored on the one or more computer readable storage media, the program instructions are executable by a computer processor to cause the computer processor to, in one embodiment: (i) perform multi-omics data pre-processing and quality control across each modality; (ii) combine modalities into higher-order tensors; and (iii) compute associations using tensor regression, wherein the computer program generates results of the tensor regression that have the potential to contribute to advancements in several areas within healthcare and biomedical research.

### EXAMPLE 1 PROOF OF CONCEPT OF USE OF TENSOR REGRESSION IN GENOME-WIDE ASSOCIATION STUDIES

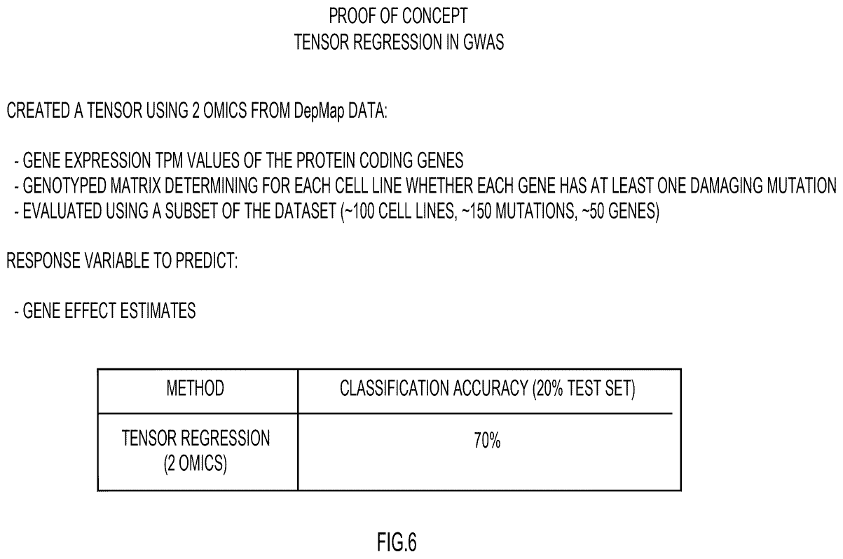

Data from DepMap (depmap.org) was used to evaluate the accuracy of the tensor regression model to predict estimates of certain gene effects in genome-wide association studies. Dual-omics data from DepMap (gene expression TPM values of protein coding genes) were regressed in a tensor regression model of the type depicted in FIG. 5. A genotyped matrix was prepared determining for each cell line whether each gene had at least one damaging mutation. Data were evaluated using a subset of the dataset (about 100 cell lines, about 150 mutations, and about 50 genes). The response variables were estimates of gene effects. Accuracy of predicting the gene effects was 70% using dual-omics data, as shown in FIG. 6. Specifically, the data used in the example are found at: depmap/org/portal/data_page?tab=currentRelease, in particular, using the following files:
1. (for gene effects): CRISPRgeneEffect.csv, providing gene effect estimates for all models, integrating using Chronos, with copy number corrected, scaled and screen quality corrected; 2. (for mutations): OmicsSomaticMutationsMatrixDamaging.csv, providing genotyped matrix determining for each cell line whether each gene has at least one damaging mutation; and 3. (for gene expression): OmicsExpressionProteinCodingGenesTPMLogp1.csv, providing gene transcripts per million values of the protein coding genes for the DepMap cell lines. It will be appreciated that the embodiments described above and illustrated in the drawings represent only a few of the many ways of implementing embodiments of the present invention.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system. These systems may include any types of monitors and input devices (e.g., keyboard, mouse, voice recognition, etc.) to enter and/or view information.

It is to be understood that the software of the present invention embodiments (e.g., analysis code 200, etc.) may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the server and/or client systems. The database systems and/or storage structures may be remote from or local to the computer or other processing systems, and may store any desired data.

The present invention embodiments may employ any number of any type of user interface (e.g., Graphical User Interface (GUI), command-line, prompt, etc.) for obtaining or providing information (e.g., analysis results etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

A report may include any information arranged in any fashion, and may be configurable based on rules or other criteria to provide desired information to a user (e.g., analysis results, etc.).

The present invention embodiments combining a tensor regression model with genome-wide association studies are not limited to the specific tasks or algorithms described above, but may be utilized with any variation on the tensor regression model example shown above in combination with any other type of association study in the healthcare or life sciences area.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for identifying associations between multi-omics data and complex diseases in genome-wide associations studies, comprising:
(i) performing, via a processor, data pre-processing and quality control across each modality;
(ii) combining, via the processor, modalities into higher-order tensors; and
(iii) computing, via the processor, associations using tensor regression, wherein the multi-omics data are analyzed through a tensor regression model to yield associations for use in genome-wide association studies to inform clinicians and researchers about complex diseases.

2. The method of claim 1, wherein the multi-omics data are derived from multiple sources, wherein at least some of the multi-omics data have high dimensionality and intricate structures, and wherein the multi-omics data are integrated through a tensor regression model for use in genome-wide association studies to gain a comprehensive understanding of complex diseases.

3. The method of claim 2, wherein the multiple sources of data are selected from the group consisting of genomics, proteomics, metabolomics, medical records, imaging records, EEG and EKG records, other human and veterinary clinical records, and other health- or life-sciences related records.

4. The method of claim 1, wherein a use of the identified associations resulting from application of tensor regression is selected from the group consisting of: identifying genetic variants associated with complex diseases; studying gene-gene interactions in relation to disease susceptibility; detecting gene-environment interactions and their impact on disease risk; uncovering associations between protein or metabolite profiles and disease outcomes; investigating biomarkers for disease diagnosis, prognosis, or treatment response; integrating data from multiple omics sources to gain a comprehensive understanding of complex diseases; identifying cross-omics associations and interactions; identifying potential drug targets by linking omics data with disease-related factors; developing personalized treatment strategies based on individual omics profiles; developing predictive models for disease risk, progression, or treatment response base on omics data; and estimating patient outcomes and prognosis.

5. The method of claim 4, wherein a use of the identified associations resulting from application of tensor regression comprises any one or more of: identifying genetic variants associated with complex diseases; identifying potential drug targets by linking omics data with disease-related factors; and developing personalized treatment strategies based on individual omics profiles.

6. The method of claim 1, wherein the tensor regression model includes:
Y=α+γ^{T}Z+ [symbol] B,X [symbol]
wherein Y represents a dependent (responsive) variable; a represents error or bias, or other components not included in the method; γ represents coefficient of vector input Z; T represents a transpose operation; Z represents a vector input; B represents a coefficient of tensor input X; X represents a tensor input; and [symbol] B,X [symbol] represents an inner product operation between the tensors,
wherein the multi-omics data are derived from multiple sources,
wherein at least some of the multi-omics data have high dimensionality and intricate structures, and
wherein the multi-omics data are integrated through a tensor regression model for use in genome-wide association studies to gain a comprehensive understanding of complex diseases.

7. The method of claim 6, further comprising performing dimensionality reduction to approximate coefficients.

8. The method of claim 7, wherein dimensionality reduction is accomplished by using sparse random projections to approximately obtain coefficients across multi-omics modalities.

9. The method of claim 6, wherein the multiple sources of data are selected from the group of areas consisting of genomics, proteomics, metabolomics, medical records, imaging records, EEG and EKG records, other human and veterinary clinical records, and other health- or life-sciences related records.

10. The method of claim 6, wherein a use of the identified associations resulting from application of tensor regression to the omics data is selected from the group consisting of: identifying genetic variants associated with complex diseases; studying gene-gene interactions in relation to disease susceptibility; detecting gene-environment interactions and their impact on disease risk; uncovering associations between protein or metabolite profiles and disease outcomes; investigating biomarkers for disease diagnosis, prognosis, or treatment response; integrating data from multiple omics sources to gain a comprehensive understanding of complex diseases; identifying cross-omics associations and interactions; identifying potential drug targets by linking omics data with disease-related factors; developing personalized treatment strategies based on individual omics profiles; developing predictive models for disease risk, progression, or treatment response base on omics data; and estimating patient outcomes and prognosis.

11. A system for identifying associations between multi-omics data and complex diseases in genome-wide associations studies, the system comprising:
one or more memories; and
a processor coupled to the one or more memories, and configured for:
(i) performing data pre-processing and quality control across each modality;
(ii) combining modalities into higher-order tensors; and
(iii) computing associations using tensor regression.

12. The system of claim 11, wherein the multi-omics data are derived from multiple sources, wherein at least some of the multi-omics data have high dimensionality and intricate structures; and wherein the multi-omics data are integrated through a tensor regression model for use in genome-wide association studies to gain a comprehensive understanding of complex diseases.

13. The system of claim 11, wherein the multiple sources of data are selected from the group consisting of genomics, proteomics, metabolomics, medical records, imaging records, EEG and EKG records, other human and veterinary clinical records, and other health- or life-sciences related records.

14. The system of claim 11, wherein a use of the identified associations resulting from application of tensor regression is selected from the group consisting of: identifying genetic variants associated with complex diseases; studying gene-gene interactions in relation to disease susceptibility; detecting gene-environment interactions and their impact on disease risk; uncovering associations between protein or metabolite profiles and disease outcomes; investigating biomarkers for disease diagnosis, prognosis, or treatment response; integrating data from multiple omics sources to gain a comprehensive understanding of complex diseases; identifying cross-omics associations and interactions; identifying potential drug targets by linking omics data with disease-related factors; developing personalized treatment strategies based on individual omics profiles; developing predictive models for disease risk, progression, or treatment response base on omics data; and estimating patient outcomes and prognosis.

15. The system of claim 11, wherein the one or more processors are further configured for performing dimensionality reduction by using sparse random projections to approximate coefficients across multi-omics modalities.

16. A computer program product for analyzing multi-omics data using tensor regression in genome-wide association studies, comprising one or more computer readable storage media having program instructions collectively stored on the one or more computer readable storage media, the program instruction executable by a computer processor to cause the computer processor to:
(i) perform multi-omics data pre-processing and quality control across each modality;
(ii) combine modalities into higher-order tensors; and
(iii) compute associations using tensor regression.

17. The computer program of claim 16, wherein the multi-omics data are derived from multiple sources, wherein at least some of the multi-omics data have high dimensionality and intricate structures, and wherein the multi-omics data are integrated through a tensor regression model for use in genome-wide association studies to gain a comprehensive understanding of complex diseases.

18. The computer program of claim 17, wherein the multiple sources of data are selected from the group consisting of genomics, proteomics, metabolomics, medical records, imaging records, EEG and EKG records, other human and veterinary clinical records, and other health-or life-sciences related records.

19. The computer program product of claim 16, wherein the instructions further cause the computer processor to perform dimensionality reduction by using sparse random projections to approximate coefficients across multi-omics modalities.
